SEQUENCE LISTING (18 sequences)

SEQ ID NO: 1 - protein, 214 aa, Mus musculus
MSCRERTDSSCGCNGHEENRILKCVVVGDGAVGKTCLLMSYANDAFPEEYVPTVFDHYAVTVTVGGKQHLLGLYDTAGQEDYNQLRPLSYPNTDVFLICFSVVNPASYHNVQEEWVPELKDCMPHVPYVLIGTQIDLRDDPKTLARLLYMKEKPLTYEHGVKLAKAIGAQCYLECSALTQKGLKAVFDEAILTIFHPKKKKKGCLGCHGCCAII

SEQ ID NO: 2 - DNA, 1846 bp, Mus musculus
ctctcctccatattagttgtgtggtggaaaaccgggcaggagttatccaaatcccaccgtgtcattagccaggctggggatggggagggtagagtgagattctcacgggctgctgatatttctagactgccaggaagtcccgggaagtctatggacatgagagtttcttaactccagctctacccccaactccacttctccgttgggattgggagaaagggacagacccttttcatccctcctcccaagagtgccttccttctccagcatggtgagctgcggggctcgagtgtgtctgggctgtgcctgggagagcccggccagccagagtcaggccgcctgagctccagcctggggccggtgccaccgctccatgcactgcagaagccccagcagctgctgcgggagccgccaacatgagctgcagagagaggaccgacagcagctgcggctgcaatggacatgaggagaacaggatcctgaagtgcgtggtggtcggggacggcgcggtggggaagacctgcttgctgatgagctacgccaacgacgccttcccagaggaatacgtgcccactgtgtttgaccactacgcagttaccgtgacggtgggaggcaagcagcacttgcttggactatacgacactgcaggacaggaggattacaaccagctgcggccactctcttaccccaacacagatgtgtttctcatctgcttctctgtcgtaaacccagcctcttaccacaacgttcaggaggagtgggtcccggaactgaaggactgcatgcctcatgtgccttatgtgctcatcggaacccagattgatctccgagatgaccccaaaaccttggcgcgtctgctgtacatgaaggagaagcccctcacctatgagcacggcgtgaagctggcgaaagcgattggagcgcagtgctacttggaatgttctgccctgacccagaagggtctcaaagcagtttttgatgaagccatcctcaccattttccaccccaagaaaaaaaagaaaggctgcttagggtgtcacggctgctgtgcaattatctgaagttgcctgacacccatctccccgccatgcaagggcatggagggcagccagtctctgtgacccaactctagccaaaaaggagggcgtgaccagaaaggaattcctgcatcccaacgcctgtcccagtgccctgccagcttcccagctcagcatcctgtaagccactgccacagtcctaagccagccagaccatgaacactgtgtatggagaatgcagagtcacattcagaccatatgctggtgaggtgttggaagccgagtgacaatccatctggtatttcatatcttctctcccacggttatggaccaacagagagtcatgacgggaaaaccaagcactctgtcctgcactggtggccttagttcatgtcttcaccaaacttgggaatacaacaccaacattttttttttctgaatcagttcgttcttttacccaagcaccttaacgctcatttctattgcagtttcccaatgtccaatcacttgagccttacaaatccacatcaaaataattgaaatacaagctggattgtgttcacaccaactgacggtggatctctgccaatgttcttagagccatcatgtacctctgaatgcctaattacaaaaaaaaaacttttctaaacttacagacctataatgtttttaaactttttaaaagaaaacatcctgtgagttacagctcccaagtttggccaaagatcatcccacaaatggttcagaagatagggggactgctcttgcaaaaccctactgttattcaaaccaaaaaaaaaaaaaaaaaa

SEQ ID NO: 3 - DNA, 22 bp, Artificial Sequence
  synthetic oligonucleotide
gtcttcgaccactacgcagtca

SEQ ID NO: 4 - DNA, 23 bp, Artificial Sequence
  synthetic oligonucleotide
gctatgatagcctcatcaaaaac

SEQ ID NO: 5 - DNA, 20 bp, Artificial Sequence
  synthetic oligonucleotide
gtgccttatgtgctcatcgg

SEQ ID NO: 6 - DNA, 20 bp, Artificial Sequence
  synthetic oligonucleotide
ctgaatgtgactctgcattc

SEQ ID NO: 7 - protein, 191 aa, Mus musculus
MQTIKCVVVGDGACGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQRGLKNVFDEAILAALEPPETQPKRKCCIF

SEQ ID NO: 8 - protein, 204 aa, Mus musculus
MAHGPGALMLKCVVVGDGAVGKTCLLMSYANDAFPEYVPTVFDHYAVSVTVGGKQYLLGDYDTAGQEDYDRLRPLSYPMTDVFDICFSVVNPASFQNVKEEWVPELKEYAPNIPFLLIGTQIDLRDDPKTLARLNDMKEKPVCVEQGQKLAKEIGACCYVECSALTQKGKLTVFDEAIIAILTPKKHTVKKRIGSRCINCCLIT

SEQ ID NO: 9 - protein, 214 aa, Mus musculus
MSCRERTDSSCGCNGHEENRILKCVVVGDGAVGKTCLLMSYANDAFPEEYVPTVFDHYAVTVTVGGKQHLLGLYDTAGQEDYNQLRPLSYPNTDVFDICFSVVNPASYHNVQEEWVPELKDCMPHVPYVLIGTQIDLRDDPKTLARLLYMKEKPLTYEHGVKLAKAIGAQCYLECSALTQKGLKAVFDEAILTIFHPKKKKKGCLGCHGCCAII

SEQ ID NO: 10 - protein, 191 aa, Homo sapiens
MQSIKCVVVGDGAVGKTCLLICYTTNAFPKEYIPTVFDNYSAQSAVDGRTVNLNLWDTAGQEEYDRLRTLSYPQTNVFVICFSLASPPSYENVRHKWHPEVCHHCPDVPILLVGTKKDLRAQPDTLRRLKEQSQAPITPQQGQALAKQIHAVRYLECSALQQDGVKEVFAEAVRAVLNPTPIKRGRSCILL

SEQ ID NO: 11 - protein, 192 aa, Mus musculus
MQAIKCVVVGDGAVGKTCLLISYTTNAFPGEYIPTVFDNYSANVMVDGKPVNLGLWDTAGQEDYDRLRPLSYPQTDVFLICFSLVSFASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 12 - protein, 192 aa, Mus musculus
MAAIRKKLVIVGDGACGKTCLLIVFSKDQFPEVYVPTVFENYVADIEVDGKQVELALWDTAGQEDYDCLRPLSYPDTVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLIL

SEQ ID NO: 13 - protein, 244 aa, Homo sapiens
MKERRASQKLSSKSIMDPNQNVKSKIVVVGDSQCGKTALLHVFAKDCFPENYVPTVFENYTASFEIDTQRIELSLWDTSGSPYYDNVRPLSYPDSDAVLICFDISRPETLDSVLKKWKGEIQEFCPNTKMDDVGCKSDLRTDVSTLVELSNHRQTPVSYDQGANMAKQIGAATYIECSALQSENSCRDIFHVATLACVNKTNKNVKRNKSQRATKRISHMPSRPELSAVATDLRKDKAKSCTVM

SEQ ID NO: 14 - protein, 232 aa, Homo sapiens
MKERRAPQPVVARCKLVLVGDVQCGKTAMLQVLAKDCYPETYVPTVFENYTACLETEEQRVELSDWDTSGSPYYDNVRPLCYSDSDAVLLCFDISRPETVDSALKKWRTEILDYCPSTRVDDIGCKTDLRTDLSTLMELSHQKQAPISYEQGCAIAKQLGAEIYDEGSAFTSEKSIHSIFRTASMLCLNKPSPLPQKSPVRSLSKRLLHLPSRSELISSTFKKEKAKSCSIM

SEQ ID NO: 15 - protein, 210 aa, Mus musculus
MNASQVAGEEAPQSGHSVKVVLVGDGGCGKTSLMMVFAKGAFPESYSPTVFERYNATLQMKGKPVHLQIWDTAGQDDYDRLRPLFYPDANVLLLCFDVTNPNSFDNVSNRWYPEVTHFCKGVPIIVVGCKIDLRKDKVLVNNLRKKRLEPVTYHRGHDMARSVGAVAYLECSARLHDNVEAVFQEAAEVALSSRRHNFWRRITQNCCLAT

SEQ ID NO: 16 - protein, 191 aa, Homo sapiens
MLSSIKCVLVGDSAVGKTSLLVRFTSETFPEAYKPTVYENTGVDVFMDGIQISLGLWDTAGNDAFRSIRPLSYQQADVVLMCYSVANHNSFLNLKNKWIGEIRSNLPCTPVLVVATQTDQREMGPHRASCVNAMEGKKLAQDVRAKGYLECSALSNRGVQQVFECAVRTAVNQARRRNRRRLFSINECKIF

SEQ ID NO: 17 - protein, 4 aa, Artificial Sequence
  consensus sequence
CXXX

SEQ ID NO: 18 - protein, 11 aa, Artificial Sequence
  consensus sequence
CXXCXXCCXXX

(19) United States
(12) Patent Application Publication
(10) Pub. No.: US 2005/0112748 A1
(43) Pub. Date: May 26, 2005

(54) Low molecular weight gtpase rhot

(76) Inventors: Takeshi Endo, Chiba-shi (JP); Tomoyuki Abe, Chiba-Shi (JP); Hiroaki Miki, Shibuya-Ku (JP); Tadaomi Takenawa, Ota-Ku (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/490,381

(22) PCT Filed: Oct. 3, 2001
(86) PCT No.: PCT/JP01/08702

(51) Int. Cl. C12N 9/22; A61K 38/46; A61K 48/00

(52) U.S. Cl. 435/199; 424/94.6; 514/44

(57) ABSTRACT

Small GTPase RhoT having an amino acid sequence represented by SEQ ID NO: 1, or an amino acid sequence derived from this amino acid sequence by the substitution, deletion, addition, or insertion of one or more amino acid residues; its gene; and drugs containing the same. RhoT has an excellent effect of forming and/or extending neurites.

### TECHNICAL FIELD

The present invention relates to RhoT and its gene. RhoT is a novel, small GTPase belonging to the Rho family and exhibits excellent neurite formation action and neurite outgrowth induction action.

### BACKGROUND ART

Rho family small GTPases are central regulators of the actin cytoskeleton and associated cell structures determining cell shape, cell migration, cell motility, cytokinesis, and cell adhesion. They also participate in signaling pathways regulating gene transcription, cell transformation, differentiation, and apoptosis. According to current knowledge, the Rho family consists of about 14 members, which are grouped into six subfamilies: Rho (RhoA, RhoB, RhoC), Rac (Rac1, Rac2, Rac3, RhoG), Cdc42 (Cdc42, Tc10), RhoE/Rnd (RhoE, Rnd1, Rnd2), RhoD, and RhoH.

Among them, RhoA, Rac1, and Cdc42 have been studied in detail. In fibroblasts, activation of RhoA by the extracellular agonist lysophosphatidic acid (LPA) leads to the assembly of contractile actin stress fibers and associated focal adhesions. By contrast, exogenously expressed constitutively active forms of RhoD, RhoE, and Rnd1 disassemble these cytoskeletal structures by antagonizing RhoA. Rac1 activated by platelet-derived growth factor or insulin induces the assembly of an actin filament meshwork to generate membrane ruffles (lamellipodia) and specific focal complexes. Cdc42 activated by bradykinin is responsible for the formation of actin filament-containing microspikes (filopodia) and associated focal complexes. In addition, Cdc42 can activate Rac1, and consequently extension of filopodia is accompanied by concerted lamellipodial spreading. Tc10, which is closely related to Cdc42, produces peripheral processes longer than the filopodia formed by Cdc42 (Curr. Biol. 8: 1151-1160 (1998)).

In neuronal cells, these small GTPases play important roles in extension of neurites and remodeling of growth cones. Clostridium botulinum C3 exoenzyme, which inactivates RhoA by ADP-ribosylating its effector domain, induces neurite outgrowth in PC12 pheochromocytoma cells and N1E-115 neuroblastoma cells. On the other hand, microinjection of constitutively active RhoA or its target protein ROCK/Rho-kinase/ROK in neurite-extending PC12 or N1E-115 cells as well as the treatment of these cells with LPA causes neurite retraction and growth cone collapse (J. Cell Biol. 141: 1625-1636 (1998)). Microinjection of Cdc42 and Rac1 facilitate the formation of filopodia and lamellipodia, respectively, at the growth cones and along neurites of N1E-115 cells (Mol. Cell Biol. 17: 1201-1121 (1997)). The growth cones of neurons guide neurites to their proper targets by constantly extending and retracting filopodia and lamellipodia (Curr, Opin. Neurobiol. 1: 339-345 (1991), Curr. Opin. Neurobiol. 4: 43-48 (1994)). Filopodia act as sensors for guiding the growth cone, whereas lamellipodia are implicated in neurite extension and cellular movement via membrane extension. Dominant-negative Cdc42(T17N) or Rac1(T17N) interferes with the neurite outgrowth induced by C3 exoenzyme or nerve growth factor (NGF). Thus, Cdc42 and Rac1 are required for the neurite outgrowth through the formation of filopodia and lamellipodia, respectively, at the growth cone (J. Biol. Chem. 274: 19901-19905 (1999), Mol. Cell Biol. 17: 1201-1211 (1997)). Dominant-negative mutants of N-WASP, which is a target protein of Cdc42 and plays essential roles in filopodium formation, prevent neurite outgrowth in PC12 and hippocampal neurons (J. Biol. Chem. 275: 11987-11992 (2000)). Despite their critical roles in neurite outgrowth, neither Cdc42 nor Rac1 is sufficient by itself for activating the signaling pathway leading to the neurite outgrowth.

Accordingly, demand exists for a substance capable of activating the signaling pathway leading to the neurite outgrowth.

### DISCLOSURE OF THE INVENTION

The present inventors have thus carried out extensive studies in search of a novel neurite outgrowth factor, and have successfully achieved cloning of the novel small GTPase RhoT, which belongs to Cdc42 subfamily. They have concluded that RhoT is a novel protein, on the basis of their finding that RhoT has features similar to those of the known protein Tc10 but induces significantly longer and thicker neurites than Tc10 does. Thus, their finding leads to completion of the invention.

Accordingly, the present invention provides the small GTPase RhoT having an amino acid sequence represented by SEQ ID NO: 1, or amino acid sequences derived from this amino acid sequence by substitution, deletion, addition, or insertion of one or more amino acid residues.

The present invention also provides the small GTPase RhoT gene coding for the amino acid sequence represented by SEQ ID NO: 1 or amino acid sequences derived from this amino acid sequence by substitution, deletion, addition, or insertion of one or more amino acid residues.

The present invention also provides a drug containing as an active ingredient the RhoT or the gene coding therefor.

The present invention also provides use of the RhoT or the gene coding therefor in manufacture of a drag.

The present invention also provides a therapeutic method on the basis of neurite formation and/or neurite outgrowth, characterized by administering an effective amount of RhoT or the gene coding therefor to a patient in need thereof.

The present invention also provides a neurite formation and/or neurite outgrowth agent, containing RhoT or the gene coding therefor.

### BRIEF DESCRIPTION OF THE DRAWINGS

FIG. 1 shows, for purposes of comparison, amino acid sequences of mouse Cdc42, mouse Tc10, mouse RhoT, human RhoG, mouse Rac1, mouse RhoA, human RhoE, human Rnd1, mouse RhoD, and human RhoH.

Amino acids at positions of more than 50% identity are shown in white on black. G1 to G4: conserved core motifs required for GTPase activity and GDP/GTP-binding. E: effector domain. Switch regions I and II, Rho insert region, and CaaX motif are also shown.

FIG. 2 shows the relationship among Rho family proteins in molecular evolution.

FIG. 3 shows Northern blotting profiles obtained by use of various types of mouse tissues, C2 cells during differentiation, and 10T1/2 cells, showing expression of Cdc42 (A), Tc10 (B), and RhoT (C). Ethidium bromide staining pattern of the agarose gel electrophoresis of the total or cytoplasmic RNAs showing the 28S and 18S rRNAs (D).

FIG. 4 shows Northern blotting profiles showing expression of Cdc4.2 (E) and Tc10 (F) during differentiation of PC12 and N1E-115 cells and corresponding patterns stained with ethidium bromide (G). FIG. 4 also shows the results of analysis by quantitative RT-PCR on expression of RhoT (H) and glyceraldehyde 3-phosphate dehydrogenase (GAPDH) (control) (I) in mouse brain, PC12, and N1E-115 cells.

FIG. 5 shows fibroblasts (Balb/3T3 cells), in which cellular processes are produced by Cdc42, Tc10, and RhoT.

FIG. 6 shows fibroblasts (10T1/2 cells), in which cellular processes are produced by Cdc42, Tc10, and RhoT.

FIG. 7 shows fluorescent microscopic images of PC12 cells transfected with the cDNA of Cdc42, Tc10, or RhoT, either wild-type (wt) or constitutively active, fused to the pEF-BOS/Myc vector.

FIG. 8 shows fluorescent microscopic images of N1E-115 cells transfected with the cDNA of Cdc42, Tc10, or RhoT, either wild-type (wt) or constitutively active, fused to the pEF-BOS/Myc vector.

FIG. 9 shows the degree of neurite extension in PC12 cells treated as in FIG. 7. The degree of extension is represented by the multiple of the diameter of the cell body. The values are the means±SD of triplicate experiments.

FIG. 10 shows the degree of neurite extension in N1E-115 cells treated as in FIG. 8.

FIG. 11 shows the results of the yeast two-hybrid interaction assay, showing the binding of Cdc42, Tc10, and RhoT to the N-WASP CRIB motif (wt: wild-type, CA: constitutively active, and DN: dominant-negative).

FIG. 12 shows the results of a pull-down assay, showing the binding of Cdc42, Tc10, and RhoT to the N-WASP CRIB motif (a: immunoblots of N-WASP, and b: SDS-PAGE of GST-small GTPases).

FIGS. 13 and 14 show prevention of Cdc42-, Tc10-, and RhoT-induced process formation by dominant-negative mutants of N-WASP in Balb/3T3 cells. FIG. 13 shows Balb/3T3 cells cotransfected with Cdc42(G12V) cDNA, Tc10(G18V) cDNA, or RhoT(G30V) cDNA in pEF-BOS/Myc vector and N-WASP(H208D) cDNA in pcDL-SRα vector. FIG. 14 shows Balb/3T3 cells cotransfected with Cdc42(G12V). cDNA, Tc10(G18V) cDNA, or RhoT(G30V) cDNA in pEF-BOS/Myc vector and N-WASPΔcof cDNA in pcDL-SRα vector.

FIGS. 15 and 16 show abrogation of Tc10- and RhoT-induced neurite extension in PC12 cells by dominant-negative mutants of N-WASP in PC12 cells. FIG. 15 shows PC12 cells cotransfected with the cDNA of Cdc42(G12V), Tc10(G18V), or RhoT(G30V) in pEF-BOS/Myc vector and N-WASP(H208D) cDNA in pcDL-SRα vector. FIG. 16 shows PC12 cells cotransfected with the cDNA of Cdc42(G12V), Tc10(G18V), or RhoT(G30V) in pEF-BOS/Myc vector and N-WASPΔcof cDNA in pcDL-SRα vector.

FIG. 17 is a graph showing percentage of the number of neurite-extending PC12 cells treated as in FIGS. 15 and 16. The degree of extension is represented by the multiple of the diameter of the cell body. The values are the means±SD of triplicate experiments.

FIGS. 18 to 21 show suppression of dbcAMP-induced neurite outgrowth in PC12 cells and serum starvation-induced neurite outgrowth in N1E-115 cells by dominant-negative mutants of Cdc42, Tc10, and RhoT. PC12 (FIG. 18) and N1E-115 cells (FIG. 19) were transfected with the cDNA of Cdc42(T17N), Tc10(T23K), or RhoT(T35N) in pEF-BOS/Myc vector or with empty pEGFP-C1 vector (mock). Ten hours after the transfection, PC12 cells were treated with 0.5 mM dbcAMP and the N1E-115 cells were shifted to 0.5% fetal bovine serum (FEBS). FIGS. 20 and 21 are graphs showing percentage of the number of neurite-extending PC12 cells and N1E-115 cells, respectively. The degree of extension is represented by the multiple of diameter of the cell body. The values are the means±SD of triplicate experiments.

### BEST MODE FOR CARRYING OUT THE INVENTION

RhoT of the present invention is (1) an amino acid sequence represented by SEQ ID NO: 1, or (2) an amino acid sequence derived from the amino acid sequence of (1) by the substitution, deletion, addition, or insertion of one or more amino acid residues.

RhoT has the conserved motifs involved in GTPase activity and GDP/GTP bonding.

Proteins having the amino acid sequence derived from the first mentioned amino acid sequence by the substitution, deletion, addition, or insertion of one or more amino acid residues fall within the scope of the invention so long as they have GTPase activity and exhibit characteristics substantially similar to RhoT having the amino acid sequence of SEQ ID NO: 1. Preferably, the mentioned modifications result in a homology of at least 80%, more preferably at least 90% with respect to the amino acid sequence of, for example, SEQ ID NO: 1.

The RhoT gene of the present invention has a nucleotide sequence coding for (1) the amino acid sequence represented by SEQ ID NO: 1, or (2) an amino acid sequence derived from the amino acid sequence of (1) by substitution, deletion, addition, or insertion of one or more amino acid residues. Examples of such a nucleotide sequence include the nucleotide sequence of SEQ ID NO: 2, or a nucleotide sequence derived therefrom by substitution, deletion, addition, or insertion of one or more amino acid residues.

Like the case of modifications of the amino acid sequence, modifications of the nucleotide sequence are also within the scope of the present invention so long as they code for a polypeptide exhibiting characteristics similar to those of the above-mentioned RhoT. Preferably, the mentioned modifications result in a homology of at least 80%, more preferably at least 90%, with respect to the nucleotide sequence of, for example, SEQ ID NO: 2.

The gene of the present invention can be obtained by preparing a cDNA library using muscle cells of vertebrates including human, for example, using mouse muscle cells, and selecting clones of interest from the library by use of probes or antibodies which are specific to the gene of the present invention [see, for example, Proc. Natl. Acad. Sci. USA. 78: 6613 (1981), Science 222: 778 (1983)].

No particular limitation is imposed on the method for screening the cDNA library for selection of the gene of the present invention, and any ordinary method may be used. Specifically, there may be employed a method, in which, with respect to a protein (RhoT) produced by cDNA, corresponding cDNA clones are selected through immunological screening making use of a specific antibody of the RhoT; a plaque hybridization method using probes that selectively bind to a DNA sequence of interest; colony hybridization; and combinations of these methods.

Through use of the gene of the present invention in combination with routine genetic engineering techniques, RhoT can be produced conveniently, in large amounts, and consistently.

Production of RhoT is in more detail summarized as follows: A recombinant DNA vector (expression vector) capable of expressing the RhoT gene in a host cell is created. The vector is transferred to a host cell so as to transform the cells. The transformants are incubated, and subsequently RhoT is collected from the resultant culture.

The above-mentioned host cells may be prokaryotic or eukaryotic. Examples of prokaryotic host cells include a broad range of routinely employed cells, such as Escherichia coli and Bacillus subtilis. Preferably, Escherichia coli, inter alia, Escherichia coli K12 can be used. Examples of eukaryotic host cells include cells from vertebrates, yeast, etc, and vertebrate cells may be COS cells, which are monkey cells (Cell 23: 175 (1981)), Chinese hamster ovary cells, and dihydrofolate reductase deficient cell lines thereof (Proc. Natl. Acad. Sci. USA. 77: 4216 (1980)). Examples of the latter group include yeast cells belonging to genus Saccharomyces.

RhoT may also be produced through a peptide synthesis method on the basis of the amino acid sequence of SEQ ID NO; 1.

Functions of RhoT will next be described with reference to the results of the Examples, which will be described hereinlater.

Rho family small GTPases are known to regulate a diversity of cellular functions through reorganization of the actin cytoskelton. Among them, Cdc42 and Tc10 induce 9 filopodia or peripheral processes in cultured cells. Tc10 was highly expressed in skeletal muscles, heart, and brain, and remarkably induced during differentiation of C2 skeletal muscle cells and neuronal differentiation of PC12 and N1E-115 cells. On the other hand, RhoT of the present invention was predominantly expressed in heart and uterus, and induced during neuronal differentiation of N1E-115 cells. Tc10 exogenously expressed in fibroblasts generated actin-filament-containing peripheral processes longer than filopodia formed by Cdc42, whereas RhoT produced much longer and thicker actin-filament-containing processes. Furthermore, both Tc10 and RhoT induced neurite outgrowth in PC12 and N1E-115 cells, but Cdc42 did not by itself. In yeast two hybrid interaction assay and pull-down assay, RhoT and Tc10, alike the case of Cdc42, were found to bind to the CRIB-motif-containing portion of N-WASP. The formation of peripheral processes and neurites by Tc10 and RhoT was prevented by the coexpression of dominant-negative mutants of N-WASP. Thus, N-WASP is essential for the process formation and neurite outgrowth induced by Tc10 and RhoT. Neuronal differentiation of PC12 and N1E-115 cells induced by dibutyryl cyclic AMP and by serum starvation, respectively, was prevented by dominant-negative Cdc42, Tc10, and RhoT. Taken together, RhoT differs from Tc10 not only in amino acid sequence, but also in neurite formation ability.

Accordingly, RhoT of the present invention or the gene coding therefor is useful as a neurite forming agent and/or a neurite outgrowth inducing agent. Moreover, the RhoT of the present invention or the gene coding therefor is useful in the therapy of pathological conditions which need extension of neurites for treatment thereof, such as Alzheimer's disease, Parkinson's disease, and spinal cord injury.

For administering the drug of the present invention to mammalian including human, the aforementioned active agent is combined with a pharmacologically acceptable carrier and processed into a drug composition of a variety of dosage forms. A preferred dosage form is injection. Examples of pharmacologically acceptable carrier include distilled water, a solubilizer, a stabilizer, an emulsifier, and a buffer. The dose of any of the produced drugs differs depending on the identity of disease, sex, body weight, etc., but a dose of 0.1 μg to 10 mg per day or thereabouts as reduced to the RhoT protein mass would be appropriate.

### EXAMPLES

The present invention will next be described in more detail by way of examples, which should not be construed as limiting the invention thereto.

A. Materials and Methods

(1) Cell Culture

Mouse C2 skeletal muscle cells (Nature 270: 725-727 (1977)) were cultured by the known method (J. Biochem. 112: 321-329 (1992)). The proliferating myoblasts were maintained at 37° C. in Dulbecco's Modified Eagle's Medium (DME) (growth medium) supplemented with 10% fetal bovine serum (FBS). To induce terminal differentiation, about 2×10⁵ cells (about 20% confluent) were plated in the growth medium on a 100 mm-dish and maintained for 16 to 24 hours, and then the medium was replaced by DME medium supplemented with 5% horse serum (HS) (differentiation medium). Myotubes developed extensively by 96 hours after the shift to the differentiation medium. Mouse Balb/3T3 fibroblasts (J. Cell. Physiol. 72; 141-148 (1968)) and mouse C3H/10T1/2 (10T1/2) fibroblasts (Cancer Res. 33: 3231-3238 (1973)) were cultured in the growth medium. Rat PC12 pheochromocytoma cells (Proc. Natl. Acad. Sci. USA 73: 2424-2428 (1976)) were maintained in DME medium containing 10% FBS and 5% HS. To induce differentiation, the medium was replaced with DME medium supplemented with 50 ng/mL NGF (2.5 S, Promega) or with 10% FBS, 5% HS, and 0.5 mM dibutyryl cyclic AMP (dbcAMP) (Sigma). Mouse N1E-115 neuroblastoma cells (Proc. Natl. Acad. Sci. USA 69: 258-263 (1972)) were maintained in the growth medium. To induce differentiation, the cells were shifted to DME medium containing 0.5% FEBS or the growth medium supplemented with 2% dimethyl sulfoxide (DMSO).

(2) cDNA Cloning and Sequence Analyses

Cytoplasmic RNA was prepared from mouse C2 myotubes by the method described previously (Cell 49: 515-526 (1987)), and poly(A)⁺ RNA was isolated by use of Oligotex-dT30 Super (Roche). A single-stranded cDNA pool was synthesized with SuperScript II RNase H(−) reverse transcriptase (Invitrogen) from 2 μg of the template poly (A)⁺ RNA primed with an oligo(dT) primer. Mouse Tc10 cDNA fragment was cloned by reverse transcription (RT)-PCR using a sense (GTCTTCGACCACTACGCAGTCA) and an antisense (GCTATGATAGCCTCATCAAAAAC) primers derived from human Tc10 cDNA sequence (Mol. Cell. Biol. 10: 1793-1798 (1990)) (DDBJ/EMBL/GenBank accession No. M31470). The amplification reaction was carried out on Zymoreactor II (Atto) with Taq DNA polymerase (Qiagen). RhoT cDNA fragment was cloned similarly with a sense (GTGCCTTATGTGCTCATCGG) and an antisense (CTGAATGTGACTCTGCATTC) primers derived from a mouse expressed sequence tag (EST) clone (accession No. AA920345). The C2 myotube cDNA library constructed in λZAPII (Oncogene 15:2409-2417 (1997)) was screened with these cDNA fragments labeled with [α-³²P]dCTP (>111 TBq/mmol, ICN Biomedicals) by using the BcaBEST labeling kit (Takara Shuzo). A 4.00 kb cDNA and 1.85 kb cDNAs containing the entire coding regions of Tc10 and RhoT, respectively, were cloned. pBluescript SK(−) phagemid containing cloned cDNAs were obtained by in vivo exision. Nucleotide sequence of the cDNAs was determined with LI-COR 4000 automated DNA sequencing system by use of SequiTherm Long-Read Cycle Sequencing Kit-LC (Epicentre Technologies). The nucleotide and amino acid sequences were analyzed with GENETYX-Mac softwares (Ver. 10.1, Software Development Co.).

(3) Northern Blotting and Quantitative RT-PCR

Cytoplasmic RNAs of cultured cells were prepared by the previously reported method (Cell 49: 515-526 (1987)). Total RNAs of mouse tissues were prepared according to the method described by Chomczynski and Sacchi (Anal. Biochem. 162: 156-159 (1987)). Northern blotting was performed by the previously reported method (Cell 49: 515-526-(1987)). Quantitative RT-PCR was performed as described previously (J. Biochem. 128: 941-949 (2000)). The amplification reaction was conducted according to a step program (95° C., 60 seconds; 58° C., 15 seconds; and 72° C., 60 seconds). The primers used for RhoT amplification were the same as those used for the cloning. The primers for glyceraldehyde 3-phosphate dehydrogenase (GAPDH) as a control were as previously described (J. Biochem. 128: 941-949 (2000)). The amount of each product reached saturation after 50 and 17 cycles of amplification, respectively. The PCR products were analyzed by agarose gel electrophoresis.

(4) Epitope-Tagging, EGFP-Tagging, and Transfection

Point mutations to generate the constitutively active mutants of Tc10(G18V) and RhoT(G30V) and the dominant-negative mutants of Tc10(T23K) and RhoT(T3SN) were introduced in the cDNAs by use of a Transformer site-directed mutagenesis kit (Clontech Laboratories, Inc.). Coding sequences of the wild-type (wt) and the mutated proteins were fused in-frame to the N-terminal Myc-tag in pEF-BOS/Myc vector. They were also ligated to pEGFP-C1 vector (Clontech). These recombinant plasmids were transfected to the cultured cells grown on glass coverslips by the calcium phosphate-mediated method as described previously (J. Biol. Chem. 271: 27855-27862 (1996)). The transiently transfected cells were processed for immunofluorescence microscopy (J. Cell Sci. 111: 1081-1093 (1998)). The fixed and permeabilized cells were incubated with the monoclonal antibody (mAb) Myc1-9E10 recognizing the Myc-tag (Mol. Cell Biol. 5: 3610-3616 (1985)) (American Type Culture Collection) and then with fluorescein isothiocyanate (FITC)-conjugated goat anti-mouse IgG (affinity-purified, Cappel). To detect actin filaments, rhodamin-phalloidin (Molecular Probes, Inc.) was added to the secondary antibody. The specimens were observed under a Zeiss Axioskop microscope.

(5) Yeast Two-Hybrid Interaction Assay

The cDNAs encoding the wild-type, constitutively active, and dominant-negative mutants of Cdc42, Tc10, and RhoT were ligated to the Gal4 DNA-binding domain of the pGBT9 vector (Clontech). A cDNA fragment encoding the N-terminal portion of N-WASP (corresponding to 1st to 275th amino acid residues) (EMBO J. 15: 5326-5335 (1996), Nature 391: 93-96 (1998)) was fused to the Gal4 activation domain of pACT2 vector (Clontech) The yeast strain Y190 was sequentially transformed with the bait and prey plasmids. Double transformants were selected on plates of minimal synthetic dropout medium lacking leucine and tryptophan (SD/-Leu/-Trp). The activation of lacz reporter gene was analyzed by β-galactosidase colony-lift filter assay.

(6) Pull-Down Assay

Coding sequences of the wild-type small GTPase were ligated in-frame to a glutathione S-transferase (GST)-tag of pGEX-2T vector (Amersham Biosciences). The GST-tagged recombinant proteins were expressed in E. coli strain XL1-Blue and affinity-purified with glutathione-Sepharose 4B (Amersham Biosciences). The cDNA encoding full-length N-WASP was fused in-frame to the hemagglutinin (HA)₃-tag of pEF-BOS/HA vector. This recombinant plasmid was transfected to Balb/3T3 cells. Twenty-four hours after the transfection, the cells were lysed with RIPA buffer (150 mM NaCl, 50 mM Tris-HCl, pH 8.0, 1% Nonidet P-40, 0.5% Na deoxycholate, and 0.1% SDS) containing 1 mM dithiothreitol, 0.1 mM phenylmethylsulfonyl fluoride, and 4 mM MgCl₂ and centrifuged for 15 minutes at 16,000×g. The prepurified GST-tagged small GTPases were loaded with either 1 mM GTPγS or GDP and reapplied to glutathione-sepharose 4B. The cell lysate was applied to the small GTPase-coupled resin and thoroughly washed with RIPA buffer. The bound proteins were eluted with 5 mM glutathione in 50 mM Tris-HCl (pH 8.0), The eluted proteins were subjected to SDS-PAGE, and then HA-tagged N-WASP was detected by immunoblotting with anti-HA-tag polyclonal antibody (pAb) (MBL), horseradish peroxidase-conjugated goat anti-rabbit IgG (Bio-Rad) as a secondary antibody, and Renaissance western blot chemiluminescence reagent Plus (NEN Life Science Products).

B. Results

(1) RhoT is a Member of the Cdc42 Subfamily

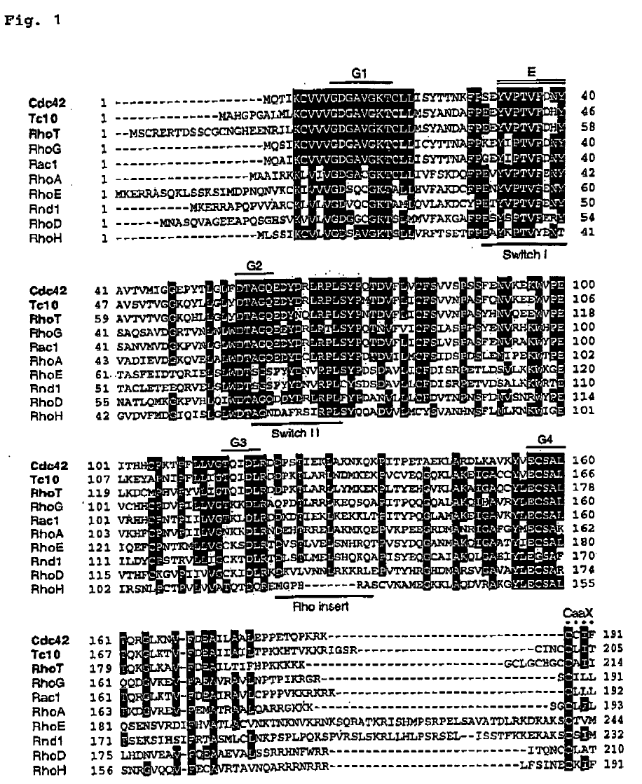

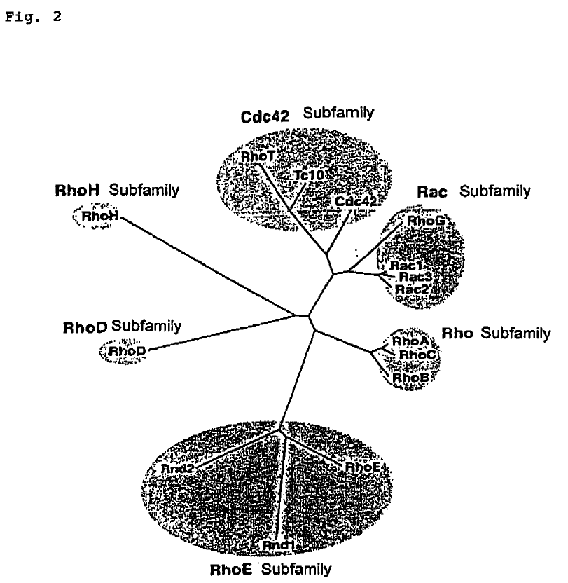

Since the mouse Tc10 cDNA sequence with the complete coding sequence had not been registered in any database, firstly RT-PCR was applied to clone its cDNA from the mouse C2 skeletal muscle myotube cDNA pool by using the primers derived from human Tc10 cDNA sequence (Mol. Cell. Biol. 10; 1793-1798 (1990)). Then the C2 myotube cDNA library was screened with the PCR product and a 3,996-bp cDNA was cloned. This mouse Tc10 cDNA contained the complete coding sequence encoding a 205-amino acid protein with a calculated Mᵣ of 22,659 (FIG. 1A). This sequence and mouse EST databases indicate that mouse Tc10 lacks the eight amino acids at the N-terminus seen in the human counterpart. A search in mouse EST databases allowed us to notice a partial cDNA sequence of mouse Tc10-like protein with the accession number AA920345. On the basis of this sequence, a fragment of this cDNA was cloned by RT-PCR from the C2 myotube cDNA pool. Using this PCR product, the C2 myotube cDNA library was screened and a 1,846-bp cDNA containing the entire coding sequence encoding a 214-amino acid protein with a calculated Mᵣ of 23,766 was obtained (FIG. 1, SEQ ID NO: 2). The amino acid sequence deduced from the nucleotide sequence had 78.6% and 64.6% identity to those of Tc10 and Cdc42, respectively. Since this protein was phylogenetically closely related to Tc10 among the Rho family proteins (FIG. 2), the inventors refer to this protein as RhoT (T comes from Tc10-homologue). RhoT, Tc10, and Cdc42 constituted the Cdc42 subfamily.

RhoT contained conserved motifs for GTPase activity and GDP/GTP binding (FIG. 1A). The switch I and II regions undergo considerable conformational change depending on the binding of GTP and GDP (Science 247: 939-945 (1990)). The sequence of the switch I region, almost corresponding to the effector domain, is relatively well conserved among each subfamily. The switch I region sequences were identical in Tc10 and RhoT. Although the sequence of the switch II region is highly conserved among Rho family proteins, RhoT contained uncharged amino acids (NQ) instead of charged amino acids (DR) in this region. The Rho insert region with 13 amino acids is specific for Rho family proteins and has been shown to participate in the binding of effector protein and RhoGDI (J. Biol. Chem. 271: 19794-19801 (1996), J. Biol. Chem. 271: 21732-21737 (1996), and J. Biol. Chem. 272: 26153-26158 (1997)). Although the sequences of this region are quite distantly related, those of Tc10 and RhoT were closely related to each other. However, two amino acids ND in this region of Tc10 were replaced by unrelated amino acids LY in that of RhoT. The C-terminal CaaX motif (C: cysteine, a: aliphatic amino acid, X: any amino acid), which is a signal for three types of posttranslational modifications, i.e., isoprenylation, proteolysis, and methylation (Annu. Rev. Cell Biol. 10: 181-205 (1994), Annu. Rev, Biochem. 65: 241-269 (1996)), was conserved in RhoT as well as in the other Rho family proteins. Tc10 and RhoT have two and three Cys residues, respectively, upstream of the CaaX motif (i.e.,—(CXX)CXXCCAPL). RhoB also has two Cys residues upstream of the CaaX motif as does Tc10. Since these Cys residues are palmitoylated in RhoB (J. Biol. Chem. 267: 20033-20038 (1992)), corresponding Cys residues of Tc10 and RhoT may also be palmitoylated. Each of Tc10 and RhoT has a very unique sequence in the N-terminal as well as in the C-terminal. In addition, RhoT had a long unique N-terminal extension as do RhoD, RhoE, and Rnd1.

(2) Tc10 and RhoT are Differentially Expressed in Muscles and Brain and Induced During Myogenic and Neuronal Differentiation.

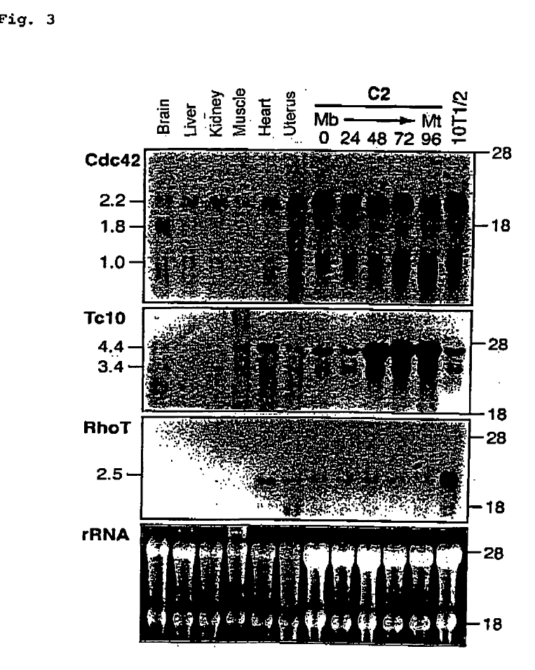

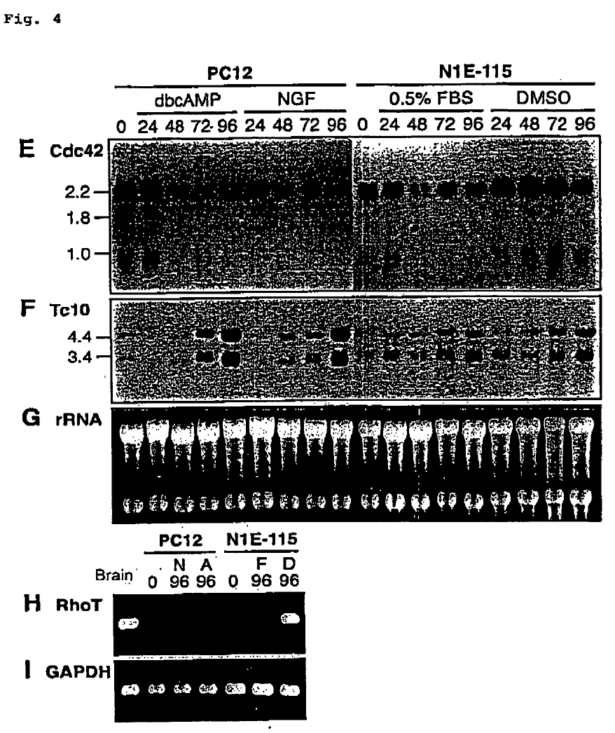

The expression levels of the Cdc42 subfamily members in tissues and cells were examined by Northern blotting. A 2.2-kb Cdc42 mRNA was ubiquitously present in a variety of mouse tissues examined, whereas a 1.8-kb mRNA was specifically expressed in the brain (FIG. 3). The amount of the 2.2-kb mRNA was almost constant throughout differentiation of C2 skeletal muscle cells and in 10T1/2 fibroblasts. In addition, a 1.0-kb mRNA was detected particularly in these cultured cells. (FIG. 3). The amount of 2.2-kb mRNA was down-regulated during neuronal differentiation of rat pheochromocytoma PC12 cells induced with NGF or dbcAMP and during differentiation of mouse N1E-115 neuroblastoma cells induced by serum starvation (FIG. 4). By contrast, it was almost unchanged during differentiation of N1E-115 cells induced with DMSO (FIG. 4),

Tc10 mRNAs (4.4 kb and 3.4 kb) were highly expressed in three types of muscle tissues, i.e., leg skeletal muscle, heart (cardiac muscle), and uterus (smooth muscle) as well as in brain (FIG. 3). The expression pattern is consistent with that in the previous reports (Curr. Biol. 8: 1151-1160 (1998), and J. Neurosci. 20; 4130-4144 (2000)). They existed at a moderate level in undifferentiated C2 myoblasts but were remarkably induced by 48 h after the induction of differentiation and remained at a high level in terminally differentiated myotubes. Further, the mRNAs were gradually induced during differentiation of PC12 cells stimulated with NGF or dbcMP and accumulated at a high level by 96 hours (FIG. 4). They were also upregulated in N1E-115 cells according to the progression of differentiation by serum starvation or by DMSO treatment (FIG. 4).

By contast, RhoT mRNA (2.5 kb) was primarily present in the uterus and heart. It also existed in skeletal muscle and brain to lesser extents (FIG. 3). Its amount was almost constant during C2 cell differentiation and higher in 10T1/2 fibroblasts than in C2 cells. The mRNA was hardly detected by Northern blotting in PC12 and N1E-115 cells regardless of their differentiated state. However, quantitative RT-PCR analyses detected RhoT mRNA in N1E-115 cells, comparable to the level in the brain, after the differentiation induced with DMSO but not by serum starvation (FIG. 4). Although the mRNA was not detected in rat PC12 cells even by the PCR analyses, this was possibly due to the inability of the used PCR primers to anneal to the rat RhoT cDNA because the primers were designed on the basis of the mouse RhoT sequence.

(3) RhoT Induces Processes Remarkably Longer and Thicker than Those Formed by Tc10

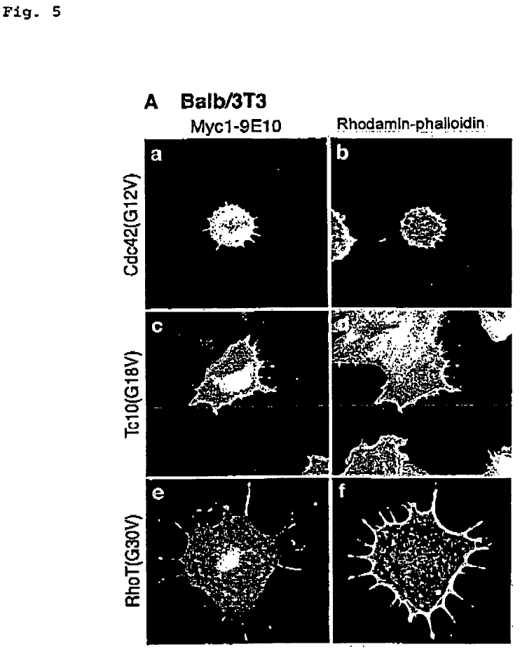

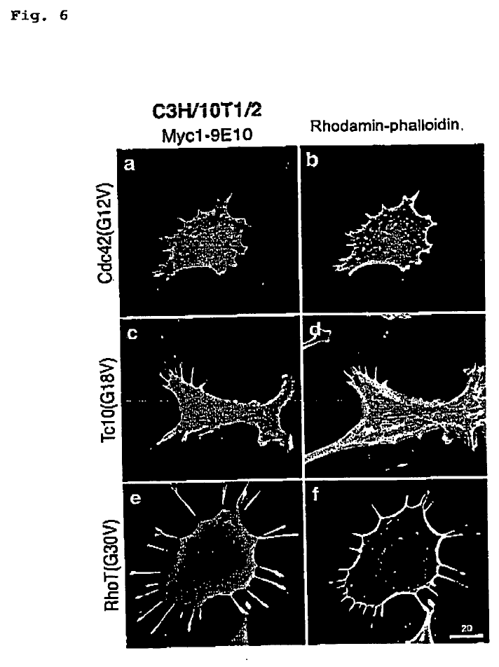

Microinjection or transfection of constitutively active Cdc642 to fibroblastic cells results in the reorganization of the actin cytoskeleton and the formation of filopodia (Mol. Cell. Biol. 15: 1942-1952 (1995), Cell 81: 53-62 (1995), J. Cell Sci. 109: 367-377 (1996)) Transfection of constitutively active Tc10 causes the disassembly of stress fibers and the formation of peripheral extensions longer than those induced by Cdc42 (Curr. Biol. 8: 1151-1160 (1998), Oncogene 18; 3831-3845 (1999)). The effects of RhoT on actin cytoskeleton and cell morphology was examined in comparison with those of Cdc42 and Tc10. Transfection of Myc-tagged constitutively active Cdc42(G12V) or Tc10(G1V) to Balb/3T3 and 10T1/2 fibroblasts caused loss of thick stress fibers and induced round cell shape and peripheral processes in both these cell types (FIGS. 5, a and c; and FIGS. 6, a and c). The processes formed by Tc10 were 10 to 20 μm long and longer than those by Cdc42 (about 10 μm long). They contained actin filaments as detected by rhodamine-phalloidin staining (FIGS. 5, b and d: and FIGS. 6, b and d). When Myc-tagged constitutively active RhoT(G30V) was expressed in these cells, stress fibers were lost, and cells assumed round shape (FIGS. 5, e and f; and FIG. 6S, e and f). RhoT (G30V) also formed actin filament-containing processes much longer (>20 μm, p sometimes as long as 40 μm) and thicker than those formed by Cdc42 or Tc10. The tips of the processes were often dilated or swollen, distinct from those formed by Cdc42.

(4) Tc10 and RhoT but not Cdc42 Induce Neurite Outgrowth

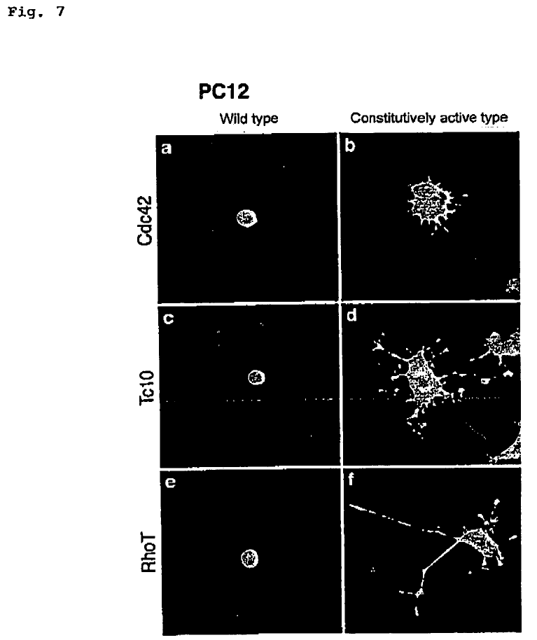

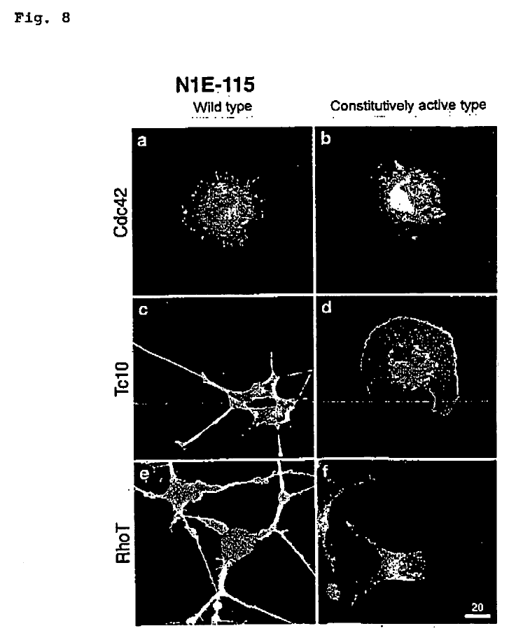

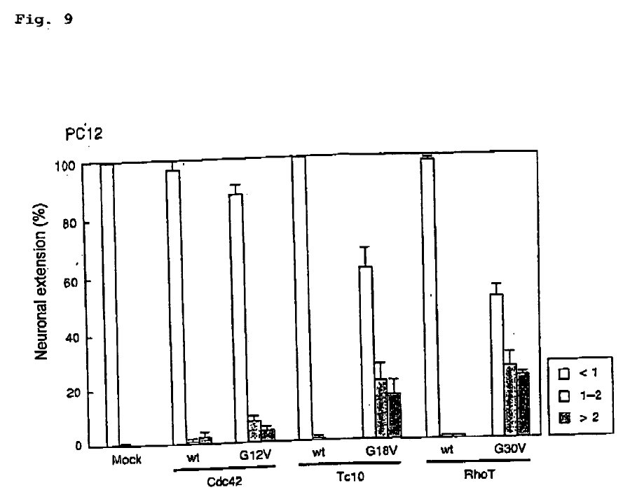

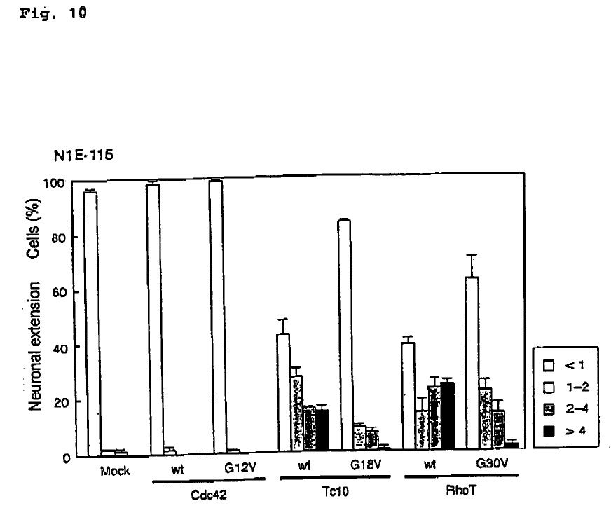

Since Tc10 and RhoT caused the formation of long processes and their mRNAs were induced during neuronal differentiation of PC12 and N1E-115 cells, the inventors next examined whether Tc10 and RhoT were responsible for the neurite outgrowth in these cells. When PC12 cells were transfected with Cdc42(G12V), filopodia were formed but neurites were barely detected (FIG. 7, b; and FIG. 9). When the cells were transfected with wild-type (wt) Cdc42, even such filopodia were rarely formed (FIG. 7a). On the other hand, Cdc42(wt) but not Cdc42(G12V) formed filopodia in N1E-115 cells (FIGS. 8, a and b). Cdc42(G12V) brought about spread or flattened forms of the cells, and neither Cdc42(wt) nor Cdc42(G12V) formed neurites in N1E-115 cells (FIGS. 8, a and b; and FIG. 10).

In contrast, both Tc10(G18V) and RhoT(G30V) induced neurites in PC12 cells, whereas their wt forms did not (FIGS. 7, c to f; and FIG. 9). RhoT(G30V) tended to form much longer neurites than did Tc10(G18V) (FIGS. 7, d and f; and FIG. 9). Both Tc10(wt) and RhoT(wt) generated long (more than four times longer than the cell body) neurites in N1E-115 cells (FIGS. 8, c and e; and FIG. 10). The neurites induced by RhoT were generally longer than those induced by Tc10. On the other hand, Tc10(G18V) and RhoT(G30V) were less effective p in giving rise to the neurites (FIGS. 8, d and f; and FIG. 10). Immunofluorescence microscopy showed that these neurites included microtubules containing tubulin and MAP2 as in the neurites formed in NGF-treated PC12 cells and in serum-starved N1E-115 cells. Dominant-negative mutants Tc10(T23K) and RhoT(T35N) were incapable of forming neutites.

(5) Tc10 and RhoT Bind to N-WASP

Cdc42 generates filopodia through binding to the CRIB motif (J. Biol. Chem. 270: 29071-29074 (1995)) of its target protein N-WASP, which activates Arp2/3-complex- and profilin-mediated actin polymerization (EMBO J. 15: 5326-5335 (1996), Nature 391: 93-96 (1998), Cell 97: 221-231 (1999)). To determine whether Tc10 and RhoT also bound to N-WASP, the yeast two-hybrid interaction assay was performed. The β-galactosidase colony-lift filter assay showed that both the wt and constitutively activer forms of Tc10 and RhoT as well as those of Cdc42 bound to the CRIB-motif-containing N-terminal portion of N-WASP (FIG. 11). By contrast, their dominant-negative forms and constitutively active RhoA(G14V) as a negative control did not bind to N-WASP.

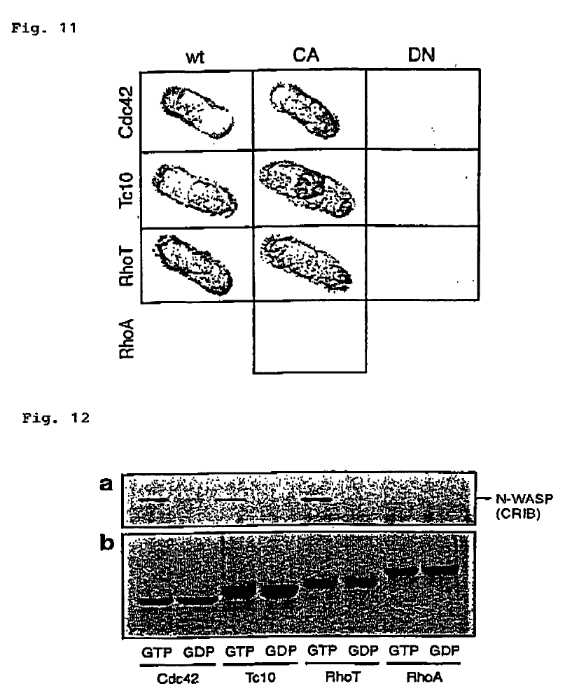

Next, the binding of these Cdc42 subfamily proteins to N-WASP was assessed by pull-down assay. GST-tagged Cdc42, Tc10, and RhoT loaded with GTPγS bound HA-tagged N-WASP expressed in Balb/3T3 cells. But the GDP-loaded Cdc42 subfamily proteins as well as GTPγS- or GDP-loaded RhoA were unable to bind N-WASP (FIG. 12). Taken together, these results imply that not only Cdc42 but also Tc10 and RhoT bind N-WASP in a GTP-dependent manner, and consequently Tc10 and RhoT also utilize N-WASP as an effector protein.

(6) Tc10 and RhoT Require N-WASP for Process Formation and Neurite Outgrowth

Since Tc10 and RhoT bound N-WASP, it is important to determine whether the binding is essential for the functions of Tc10 and RhoT. The substitution of Asp for His208 (H208D) in the CRIB motif of N-WASP abolishes the binding of Cdc42 to N-WASP (Nature 391:93-96(1998)). The cofilin homology domain of N-WASP in combination with the adjacent acidic domain participates in the binding of Arp2/3 complex to polymerize actin (Cell 97: 221-231 (1999), and J. Cell Sci. 114: 1801-1809 (2001)). The four amino acid deletion in this region (Δcof) abrogates the ability to activate Arp2/3 complex (J. Biol. Chem. 275: 11987-11992 (2000), and Cell 97: 221-231 (1999)). Since both these mutants serve as dominant-negative mutants of N-WASP (J. Biol. Chem. 275: 11987-11992 (2000)), they were used to examine the involvement of N-WASP in the process formation and neurite outgrowth by Tc10 and RhoT.

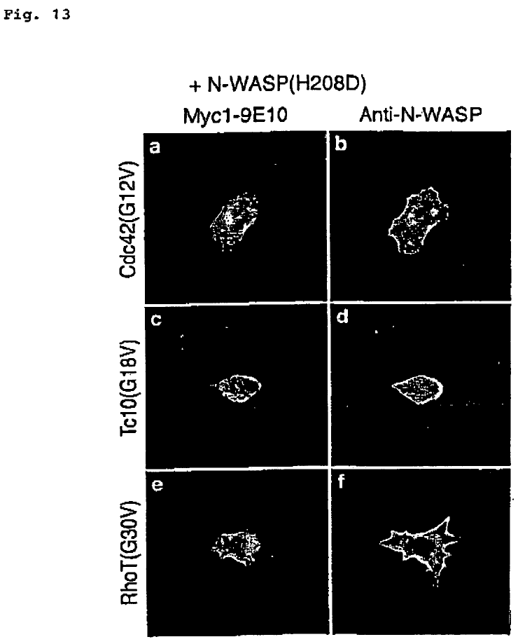

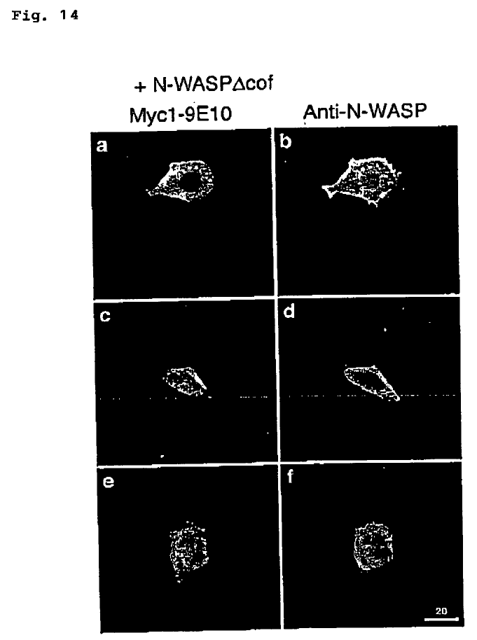

When N-WASP(9208D) or N-WASPΔcof was coexpressed with Cdc42(G12V) in Balb/3T3 cells, filopodium formation was prevented (FIGS. 13 and 14; a and b). These results are consistent with the previous observations using COS-7 cells (Nature 391: 93-96 (1998)). Coexpression of either of these N-WASP mutants with Tc10(G11V) also suppressed the process formation by Tc10 (FIGS. 13 and 14; c and d). Each of these N-WASP mutants coexpressed with RhoT(G30V) interfered with the RhoT-induced long and thick process formation as well (FIGS. 13 and 14; e and f). Similar results were obtained with 10T1/2 cells. These results indicate that N-WASP is required for the Tc10- and RhoT-mediated process formation as well as for the filopodium formation induced by Cdc42.

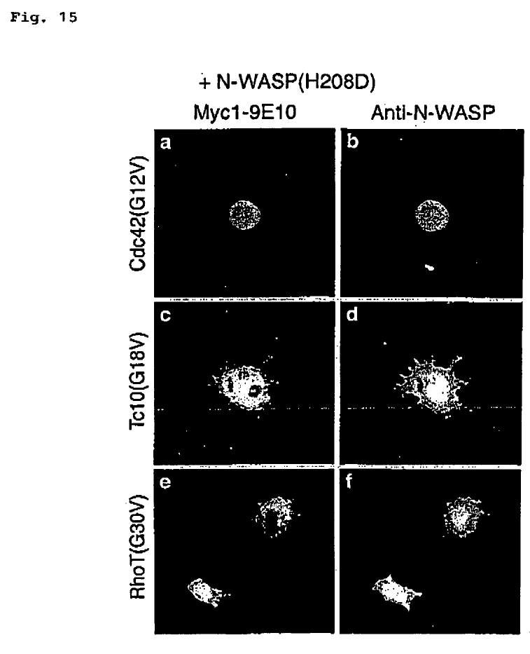

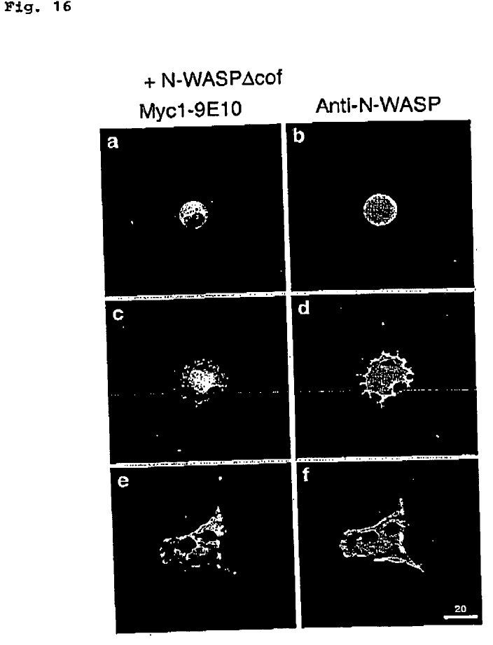

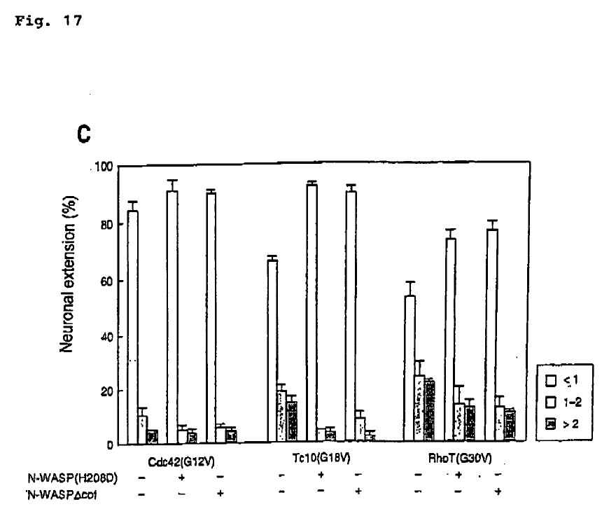

Coexpression of N-WASP(H208D) or N-WASPΔcof with Cdc42(G12v) in PC12 cells also resulted in the abrogation of filopodium formation (FIGS. 15 and 16; a and b). Furthermore, Tc10(G18V)-induced neurite outgrowth was hindered by the coexpression of N-WASP(H208t) or N-WASPΔcof (FIGS. 15 and 16, c and d; FIG. 17). Similarly, RhoT(G30V)-induced neurite outgrowth was retarded by the coexpression of the dominant-negative mutants of N-WASP (FIGS. 15 and 16, e and f; FIG. 17). These dominant-negative mutants of N-WASP also interfered with the neurite outgrowth in N1E-115 cells induced by Tc10 or RhoT. Thus, N-WASP plays essential roles in the neurite extension caused by Tc10 or RhoT as well as in the process formation in fibroblasts.

(7) Tc10 and RhoT are Essential for Neuronal Differentiation of PC12 and N1E-115 Cells

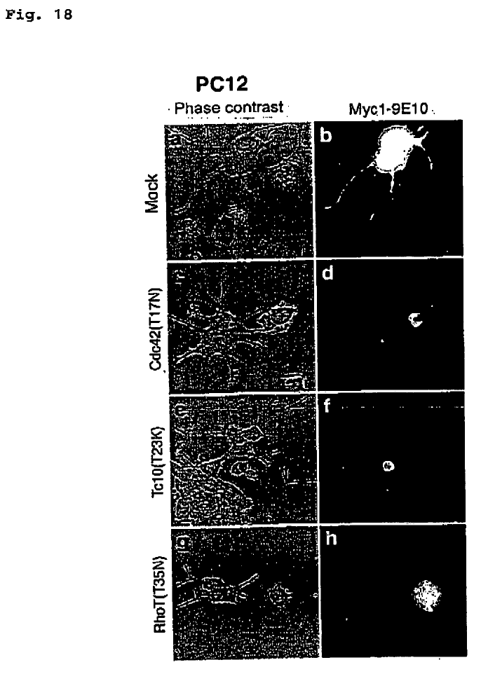

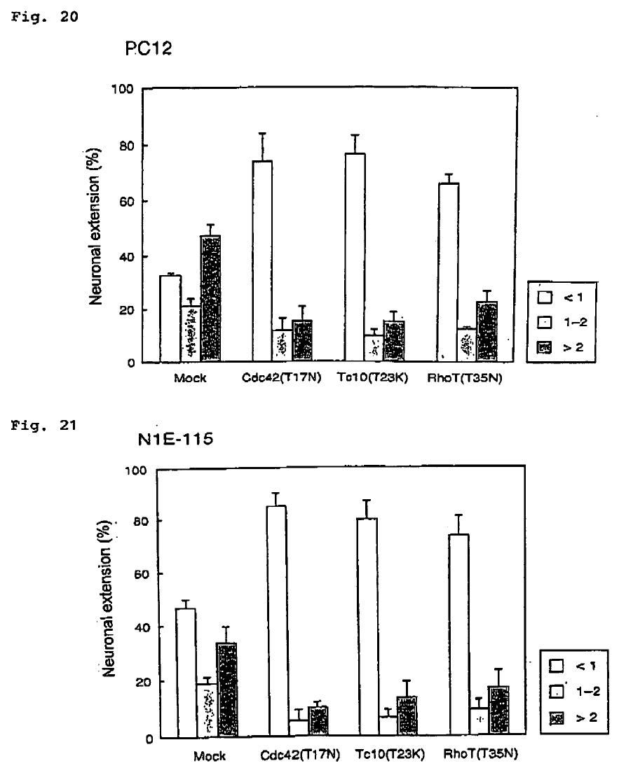

Next, the present inventors investigated whether the Cdc42 subfamily proteins were required for the neuronal differentiation in PC12 and N1E-115 cells represented by neurite extension. Differentiation of PC12 cells was induced by dbcAMP stimulation (see FIGS. 18, a, c, e, and g). Expression of dominant-negative Cdc42(T17N) in dbcAMP-stimulated PC12 cells prevented the neurite outgrowth (FIGS. 18, c and d, and FIG. 20), whereas mock transfection of the empty vector had no effect on the neurite extension (FIGS. 18, a and b). Moreover, the expression of dominant-negative Tc10(T23K) or RhoT(T35N) impeded the neurite outgrowth as well (FIGS. 18, e to h, and FIG. 20).

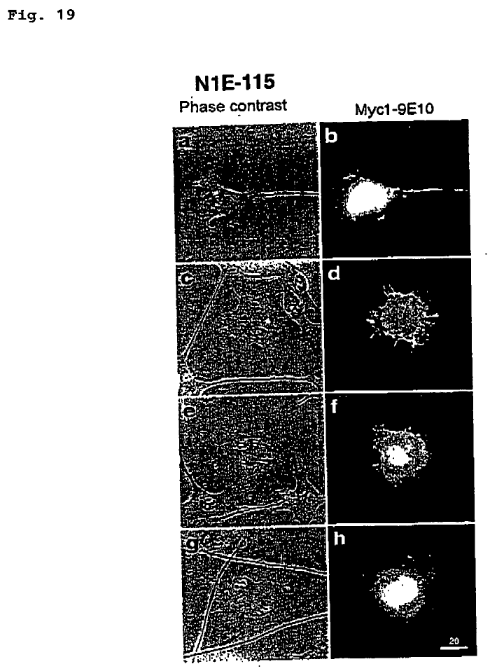

Differentiation of N1E-115 cells was induced by serum starvation (see FIGS. 19a, c, e, and g). The dominant-negative Cdc42(T17N) expressed in serum starved N1E-115 cells prevented the neurite outgrowth (FIGS. 19, c and d, and FIG. 21), although mock transfection of the empty vector did not affect the neurite extension (FIGS. 19, a and b). These results are consistent with the previous results (Kozma et al., 1997). Furthermore, the expression of dominant-negative Tc10(T23K) or RhoT(T35N) hindered the neurite outgrowth as well (FIGS. 19, e to h, and FIG. 21). These results, together with those of transfection of the wt or constitutively active mutants, imply that Tc10 and RhoT are required for neurite extension in both PC12 and N1E-115 cells.

### Industrial Applicability

RhoT of the present invention exhibits excellent neurite formation action and/or neurite outgrowth action, and thus is useful as a drug for gene therapy and regenerative therapy for neural diseases, such as Alzheimer's disease, Parkinson's disease, and spinal cord injuries.

1. Small GTPase RhoT having an amino acid sequence of SEQ ID NO: 1, or an amino acid sequence derived from the amino acid sequence of SEQ ID NO: 1 by substitution, deletion, addition, or insertion of one or a plurality of amino acid residues.

2. The small GTPase RhoT gene coding for an amino acid sequence of SEQ ID NO: 1, or for an amino acid sequence derived from the amino acid sequence of SEQ ID NO: 1 by substitution, deletion, addition, or insertion of one or a plurality of amino acid residues.

3. The RhoT gene according to claim 2, which has a nucleotide sequence of SEQ ID NO: 2, or a nucleotide sequence derived form the nucleotide sequence of SEQ ID NO: 2 by substitution, deletion, addition, or insertion of one or a plurality of bases.

4. A drug containing as an active ingredient the RhoT as recited in any one of claims 1 to 3 or a gene coding therefor.

5. The drug according to claim 4, which is a neurite forming and/or extending drug.

6. Use of the RhoT as recited in any one of claims 1 to 3 or a gene coding therefor in manufacture of a drug.

7. The use according to claim 6, wherein the drug is a neurite forming and/or extending drug.

8. A therapeutic method on the basis of neurite formation and/or neurite outgrowth, characterized by administering an effective amount of the RhoT as recited in any one of claims 1 to 3 or a gene coding therefor to a patient in need thereof.

9. A neurite forming and/or extending agent containing the RhoT as recited in any one of claims 1 to 3 or a gene coding therefor.
